(19) United States
(12) Patent Application Publication
(10) Pub. No.: US 2007/0104694 A1
(43) Pub. Date: May 10, 2007

(54) Breast stimulation and augmentation system

(76) Inventors: Rodolfo C. Quijano, Laguna Hills, CA (US); Hosheng Tu, Newport Beach, CA (US); Kenneth J. Williams, Brawley, CA (US); Robert L. Carter, Joplin, MO (US); Alexander Kiselyov, Del Mar, CA (US)

Correspondence Address: HOSHENG TU, 15 RIEZ, NEWPORT BEACH, CA, 92657-0116, US

(21) Appl. No.: 11/414,860

(22) Filed: May 1, 2006

Related U.S. Application Data
(63) Continuation-in-part of application No. 11/268,432, filed on Nov. 7, 2005.

(51) Int. Cl. A61K 35/12; C12N 5/08

(52) U.S. Cl. 424/93.7; 435/440; 435/366

(57) ABSTRACT

A stem-cell-seeded porous scaffold implant and electromagnetic growth stimulation for treating or augmenting a breast tissue defect in a patient.

### CROSS-REFERENCE TO RELATED APPLICATIONS

This application is a continuation-in-part application of U.S. patent application Ser. No. 11/268,432, filed Nov. 7, 2005, entitled “Breast Augmentation System,” the entire contents of the co-pending application are incorporated herein by reference.

### FIELD OF THE INVENTION

The present invention is related to stimulation and cells therapy for treatment of breast tissue, more particularly, the present invention relates to physical stimulation or cell-seeded implant and delivery system thereof to repair or augment a breast tissue defect in a patient.

### BACKGROUND OF THE INVENTION

The normal development of multicellular organisms relies on the orchestrated regulation of when, where, and how each cell proliferates. The formation of the intricate anatomical features of internal organs or the proper migration of nerves throughout the body require that each participating cell senses its environment and respond appropriately to developmental cues. The requirement for regulated proliferation is equally important for the proper functioning of the mature multicellular organism. A large number of replacement cells must be produced daily. The number and type of cells that are induced to proliferate as replacements depends upon the circumstances under which the original cells were eradicated and the tissues affected.

Managing the body's ability to regulate spatial and temporal aspects of cell proliferation is one approach to treating diseases and conditions characterized by traumatic or pathogenic tissue destruction. Growth factors have been considered candidate therapeutics for treating a number of such conditions because they are synthesized by and stimulate cells required for tissue repair, and are deficient in a number of chronic conditions. With the understanding that defects in growth factor signaling contribute to the development and/or persistence of a number of chronic conditions, it is logical to conclude that reinstitution or normalization of that signaling would promote healing. Although there is some evidence that pharmacological application of growth factors enhances healing in some conditions such as wound repair, it is often difficult to achieve targeted delivery of growth factors in such a way that healthy tissues are not inadvertently stimulated.

It is known that stimulation of cells with electromagnetic energy modulates the activity of genes involved in tissue repair and cell growth/proliferation and the cellular levels of gene products that are involved in molecular regulatory networks. Further, stimulation with electromagnetic energy modulates the levels of gene products such as extracellular matrix receptors, signal transduction proteins, cell cycle regulators, transcription factors and nucleic acid synthesis proteins. The changes to these regulatory networks lead to changes in cellular functions that include acceleration of the cell cycle, stimulation of wound healing, stimulation of cell proliferation, stimulation of tissue growth, and modulation of inflammatory responses. One aspect of the invention provides methods for delivering to a cell an effective amount of electromagnetic energy to change such cellular functions.

It was reported that adipose-derived stem cells might be engulfed in injured heart muscle following a heart attack-like injury. Adipose, also known as fat tissue, contains a specialized class of stem cells, which are comprised of multiple cell types that might promote healing and repair. It appears that adipose-derived stem cells home in on specific sites of injury through biological signaling that occurs naturally during heart attacks.

In addition to pluripotent stem cells of embryonic origin, several groups described mammalian multipotent stem cell populations that are obtained from adult somatic cell sources. Non-embryonic multipotent stem cells include, for example, neural stem cells, mesenchymal stem cells, bone marrow stem cells and stem cells obtained from liposuction. It is important to note that the adult multipotent stem cells described in the prior art have limited potential, in that they have not been demonstrated to give rise to any and all cell types of the body. In general, a stem cell shows ability of a clonal stem cell population to self-renew, ability of a clonal stem cell population to generate a new, terminally differentiated cell type in vitro and ability of a clonal stem cell population to replace an absent terminally differentiated cell population when transplanted into an animal depleted of its own natural cells.

Mesenchymal stem cells are adult multipotent cells derived from multiple sources, including bone marrow stroma, blood, dermis, and periosteum. These cells can be cultured continuously in vitro without spontaneous differentiation. However, under the proper conditions, mesenchymal stem cells can be induced to differentiate into cells of the mesenchymal lineage, including adipocytes, chondrocytes, osteocytes, tenocytes, ligamentogenic cells, myogenic cells, bone marrow stroma cells, and dermogenic cells (U.S. Pat. No. 5,736,396). It was reported that mesenchymal cells, upon injection into either mouse or rat brains, are capable of migrating through the brain, engrafting, surviving, and differentiating into astrocytes, ependymal cells, or neurons, suggesting the capacity of mesenchymal stem cells to give rise to cells of a non-mesenchymal lineage (U.S. Pat. No. 5,197,985, No. 5,226,914, No. 5,486,359, and No. 5,736,396).

U.S. Pat. Nos. 6,429,013 and 6,841,150, entire contents of which are incorporated herein by reference, discloses pluripotent stem cells generated from adipose tissue-derived stromal cells and uses thereof. Specifically, the patents disclose that an isolated adipose tissue derived stromal cell is induced to express at least one characteristic of a neuronal cell, an astroglial cell, a hematopoietic progenitor cell, and a hepatic cell. Further, the patents discloses a method for dedifferentiating isolated adipose tissue-derived stromal cells, comprising: plating the isolated adipose tissue-derived stromal cells at a density of approximately 1,000 to 500,000 cells/cm² and incubating the cells in medium comprising i) serum; ii) at least one compound selected from the group consisting of: growth factors, hormones, cytokines and serum factors; and iii) optionally, an embryonic extract.

Important parts of the breasts include mammary glands, the axillary tail, the lobules, Cooper's ligaments, the areola and the nipple. As breasts are mostly composed of adipose tissue, their size can change over time if the woman gains or loses weight. Adipose tissue is an anatomical term for loose connective tissue composed of adipocytes. Its main role is to store energy in the form of fat, although it also cushions and insulates the body. It has an important endocrine function in producing hormones such as leptin, resistin and TNFα. It also functions as a reservoir of nutrients. Adipose tissue has an “intracellular matrix,” rather than an extracellular one. Adipose tissue is divided into lobes by small blood vessels. The cells of this layer are adipocytes.

Recent advances in biotechnology have allowed for the harvesting of adult stem cells from adipose tissue, allowing stimulation of tissue regrowth using a patient's own cells. The use of a patient's own cells reduces the chance of tissue rejection.

Five stages of breast development include: a) the first childhood stage: the breasts are flat and show no signs of development; b) the second breast bud stage: milk ducts and fat tissue form a small mound; c) the third breast growth stage: breast become rounder and fuller; d) the fourth stage with nipple and areola forming separate small mound: not all girls go through this stage; and e) the firth stage: breast growth enters final stage showing an adult breast full and round shaped. For those women with breast defect, it is desirable to transplant stem cells or stem-cell-seeded porous scaffold as an implant to repair or augment the breast tissue defect.

Whereas embryonic stem cells are the building blocks for all of the cell types in the body, adult stem cells are a more specialized type of progenitor cell. Adult stem cells are found in specific tissues and have the ability to regenerate themselves, as well as differentiate into all of the cell types found in that tissue. The specific differentiation pathway that these cells enter depends upon various influences from mechanical influences and/or endogenous bioactive factors, such as growth factors, cytokines, and/or local microenvironmental conditions established by host tissues. Using cells from the developed individual, rather than an embryo, as a source of autologous or allogeneic stem cells would overcome the problem of tissue incompatibility associated with the use of transplanted embryonic stem cells, as well as solve the ethical dilemma associated with embryonic stem cell research

Adipose tissue offers a potential source of multipotential stromal stem cells. Adipose tissue is readily accessible and abundant in many individuals. Obesity is a condition of epidemic proportions in the United States, where over 50% of adults exceed the recommended BMI based on their height. Adipocytes can be harvested by liposuction on an outpatient basis. This is a relatively noninvasive procedure with cosmetic effects that are acceptable to the vast majority of patients. It is well documented that adipocytes are a replenishable cell population. Even after surgical removal by liposuction or other procedures, it is common to see a recurrence of adipocytes in an individual over time. This suggests that adipose tissue contains stromal stem cells that are capable of self-renewal.

Clearly, there remains a need to develop systems and methods whereby biological activities of cells, such as, but not limited to cell growth, can be stimulated by direct application of electromagnetic stimulation. In view of the foregoing, an object of this invention is to provide electromagnetic growth stimulation as a potential tool to repair or augment a breast tissue defect in a patient.

### SUMMARY OF THE INVENTION

One object of the invention is to provide a method and compositions for directing adipose-derived stromal cells cultivated in vitro to differentiate into breast tissue progenitor cells for implantation into a recipient for the therapeutic treatment of pathologic conditions in breast tissue.

Some aspects of the invention relate to a method of providing stem cells for treatment of breast tissue defect. In one preferred embodiment, the method comprises providing stem-cell-seeded porous scaffold or construct as an implant to repair or augment a breast tissue defect in a patient. The adipose-derived stem cells home in on specific sites of breast defect or injury through biological signaling that occurs naturally for a breast defect or pathologic conditions.

Some aspects of the invention relate to a method of providing stem cells for cosmetically modifying breast tissue, wherein the method comprises providing stem-cell-seeded scaffold or construct as an implant to cause breast tissue defect due to implantation and providing breast tissue regeneration through stem cells of stem cell-seeded scaffold or construct for repairing or augmenting the breast tissue defect in a patient.

Some aspects of the invention relate to a method of treating a breast defect in a patient, the method comprising differentiating an isolated human adipose tissue derived stromal cell into a breast tissue progenitor cell and administering the breast tissue progenitor cell to a breast defect area in the patient. In one embodiment, the progenitor cell further comprises a biocompatible shaped matrix or scaffold, wherein the biocompatible matrix may be non-biodegradable or biodegradable. In a further embodiment, the biodegradable matrix may be made of a material selected from a group consisting of polymers or copolymers of lactide, glycolide, caprolactone, polydioxanone, trimethylene carbonate, polymers or copolymers of polyorthoesters and polyethylene oxide, and polymers or copolymers of aliphatic polyesters, alginate, cellulose, chitin, chitosan, collagen, copolymers of glycolide, copolymers of lactide, elastin, fibrin, glycolide/l-lactide copolymers (PGA/PLLA), glycolide/trimethylene carbonate copolymers (PGA/TMC), glycosaminoglycans, and hydrogel. In a further embodiment, the biocompatible matrix comprises a material selected from a group consisting of alginate, agarose, fibrin, collagen, methylcellulose, and combinations thereof.

In one embodiment, the breast defect is traumatically created by any of the following conditions or processes: inserting the biocompatible matrix into the patient, lumpectomy, mastectomy, breast reconstruction, breast injury, or other breast surgical procedures.

In an alternative embodiment, the progenitor cell further comprises a biocompatible cell carrier, wherein the cell carrier may be in a form selected from a group consisting of slurry, gel, colloid, solution, or suspension that is flowable. In one embodiment, the cell carrier or gel is malleable. Further, the cell carrier is selected from a group consisting of alginate, agarose, fibrin, collagen, chitosan, gelatin, elastin, and combinations thereof. In one embodiment, the biocompatible cell carrier is biodegradable.

Some aspects of the invention relate to a method of treating a breast defect in a patient, the method comprising differentiating an isolated human adipose tissue derived stromal cell into a breast tissue progenitor cell and administering the breast tissue progenitor cell to a breast defect area in the patient, wherein following administration of the progenitor cell to a breast defect area in the patient, the progenitor cell further differentiates in situ in the patient.

Some aspects of the invention provide a composition for treating a breast defect of a patient, comprising stem cells derived from adipose tissue and a temperature-sensitive cell carrier, wherein the stem cells may comprise breast tissue progenitor cells. In one embodiment, the temperature-sensitive cell carrier is methylcellulose, poly(N-isopropyl acrylamide), or the like. In one embodiment, the temperature-sensitive cell carrier is characterized by a first solution phase at a lower temperature and a second gel phase at a higher temperature. In another embodiment, the temperature-sensitive cell carrier is characterized. by an expanded conformation at a lower temperature and a collapsed conformation at a higher temperature. In a further embodiment, the composition is a compressible foam, a shaped scaffold, a porous matrix or flowable/malleable material.

Some aspects of the invention provide a breast matrix system for treating a breast defect of a patient, comprising an implantable breast matrix and stem cells component, wherein stem cells are derived from adipose tissue. In one embodiment, the breast matrix comprises a fishbone configuration, the fishbone-configured breast matrix being characterized by an expandable construct with a plurality of close cells formed between longitudinal elements and connecting transverse elements. In another embodiment, the breast matrix system further comprises a delivery instrument for delivering the fishbone-configured breast matrix to a breast of the patient for treating the breast defect.

In one embodiment, the breast matrix of the breast matrix system of the present invention comprises an umbrella configuration, the umbrella-configured breast matrix being characterized by a plurality of radially expandable extending elements, each extending element having a distal end and a proximal end, wherein the proximal ends from all extending elements are secured together at one point. In another embodiment, the breast matrix system further comprises a delivery instrument for delivering the umbrella-configured breast matrix to a breast of the patient for treating the breast defect.

In one embodiment, the breast matrix of the breast matrix system of the present invention comprises a wrap-around configuration, the wraparound-configured breast matrix being made of shape memory material and characterized by a first pre-implant low-profile configuration at a lower temperature and a second implanted configuration at a higher temperature. In a further embodiment, the shape memory material is biodegradable polymer or Nitinol.

In one embodiment, the breast matrix of the breast matrix system of the present invention comprises a yo-yo configuration, the yoyo-configured breast matrix being characterized by a plurality of circular rings with varying diameters, wherein at least two circular rings are releasably secured to each other by a circular semi-ring to form an overall bowl-like configuration.

In one embodiment, the breast matrix is biodegradable or bioresorbable. In another embodiment, the breast defect is traumatically created by a process of inserting the breast matrix into a breast of the patient. In still another embodiment, the stem cells portion comprises breast tissue progenitor cells.

In one embodiment, the breast matrix system further comprises a medium for containing the stem cells or breast tissue progenitor cells. In another embodiment, the medium comprises at least one growth factor selected from a group consisting of transforming growth factor-β, insulin-like growth factor, platelet derived growth factor, epidermal growth factor, acidic fibroblast growth factor, basic fibroblast growth factor, and hepatocytic growth factor. In still another embodiment, the medium comprises at least one nutrient selected from a group consisting of vitamin A, retinoic acid, vitamin B series, and vitamin C.

Some aspects of the present invention provide a delivery instrument for delivering an umbrella-configured breast matrix to a breast of a patient comprising: a hollow tubular sheath having a distal tip, a lumen having an opening at the distal tip, and a handle portion; a plunger inside the lumen, wherein the plunger is activated by a pushing mechanism located at the handle portion, and wherein the lumen is sized and configured for appropriately receiving an umbrella-configured breast matrix at a collapsed profile.

Some aspects of the invention provide a delivery instrument for delivering an umbrella-configured breast matrix to a breast of a patient comprising a tubular applicator having a distal tip, a distal portion and a handle portion, wherein the distal portion is sized and configured for appropriately receiving an umbrella-configured breast matrix at a collapsed profile over the distal portion. In one embodiment, the umbrella-configured breast matrix is characterized by a plurality of radially expandable extending elements, each extending element having a distal end and a proximal end, wherein the proximal ends from all extending elements are secured together at one point, and wherein the umbrella-configured breast matrix further comprises at least one connecting member between any two extending elements, wherein the connecting member is selected from a group consisting of netting, strings, threads, porous membranes, and porous biodegradable films.

Some aspects of the invention provide a method for stimulating growth of administered cells in a breast, comprising the steps of: (a) administering a population of stem cells to the breast of a subject, wherein the stem cells are derived from adipose tissue; and (b) delivering to the population an effective amount of electromagnetic energy from an electromagnetic energy generator to stimulate growth of the population. In one embodiment, the electromagnetic energy is delivered with an electromagnetic field or a pulsed electromagnetic field.

Some aspects of the invention provide a method for stimulating growth of administered cells in a breast, comprising the steps of: (a) administering a population of cells to the breast of an individual; and (b) delivering to the population an effective amount of electric energy via capacitative coupling stimulation means for stimulating growth of the population. In one embodiment, the capacitative coupling stimulation means is delivered from a treatment applicator mounted on a bra or breast support.

Some aspects of the invention provide a method for stimulating one or more biological activities of cells comprising contacting the cells with an electroactive substrate, wherein the electroactive substrate comprises a breast implant made of conductive material or conductive micro/nanoparticles.

### BRIEF DESCRIPTION OF THE DRAWINGS

Additional objects and features of the present invention will become more apparent and the disclosure itself will be best understood from the following Detailed Description of the Exemplary Embodiments, when read with reference to the accompanying drawings.

FIG. 1 shows a schematic diagram of a method for treating a breast defect.

FIG. 2 shows an anatomic illustration of a woman breast.

FIG. 3 shows a breast implant embodiment of the fishbone design; (A) an expanded profile, and (B) a collapsed profile.

FIG. 4 shows a first breast implant embodiment of the umbrella design; (A) a delivery instrument, (B) an expanded device profile, and (C) a collapsed device profile.

FIG. 5 shows a second breast implant embodiment of the umbrella design; (A) a delivery instrument, (B) a proximal cross-sectional view, (C) a distal cross-sectional view, and (D) an expanded device profile.

FIG. 6 shows a breast implant of the wrap-around design; (A) an expanded profile, (B) a collapsed profile, and (C) a simulated profile.

FIG. 7 shows a breast implant of the yo-yo design.

FIG. 8 depicts the effect of an external stimulus on cells.

FIG. 9 shows a bra apparatus having capability for electromagnetic stimulation ftunctions.

### DETAILED DESCRIPTION OF THE EXEMPLARY EMBODIMENTS

The preferred embodiments of the present invention described below relate particularly to methods and a composition for the differentiation and culture of adipose tissue-derived stromal cells into breast tissue progenitor cells. The cells produced by the methods of the invention are useful in providing a source of fully differentiated and functional cells for tissue regeneration for the treatment of human breast defect, repair and augmentation. Thus, in one aspect, the invention provides a method for differentiating adipose tissue-derived stromal cells into breast tissue progenitor cells comprising culturing stromal cells in a composition that comprises a medium capable of supporting the growth and differentiation of stromal cells into functional progenitor cells.

This invention further provides methods for the introduction and position of these stromal cells in breast defect areas for repair or augmentation. While the description sets forth various embodiment specific details, it will be appreciated that the description is illustrative only and should not be construed in any way as limiting the invention. Furthermore, various applications of the invention, and modifications thereto, which may occur to those who are skilled in the art, are also encompassed by the general concepts described below.

It is known that biophysical inputs, including electric and electromagnetic fields, regulate the expression of genes in connective tissue cells for structural extracellular matrix proteins resulting in an increase in cartilage and bone production (R K Aaron et al., Clin Orthop. 2004 February:30-37). In in vivo models and clinical situations, this stipulates as enhanced repair and a gain in mechanical properties of the repairing tissues. Biophysical interactions of electric and electromagnetic fields at the cell membrane with respect to transmembrane signaling, channel activation, growth factor stimulation, and receptor stimulation or blockade are not well understood. Nevertheless, electric and electromagnetic fields increase gene expression for, and synthesis of, growth factors and this may function to amplify field effects through autocrine and paracrine signaling. In one example, electric and electromagnetic fields can produce a sustained upregulation of growth factors, which enhance, but do not disorganize endochondral bone formation.

By “progenitor” it is meant an oligopotent or multipotent stem cell which is able to divide without limit and, under specific conditions, can produce daughter cells which terminally differentiate such as into breast cells. These cells can be used for transplantation into a heterologous, autologous, or non-autologous host. By heterologous is meant a host other than the animal from which the progenitor cells were originally derived. By autologous is meant the identical host from which the cells were originally derived. Cell suspensions in culture medium are supplemented with certain specific growth factor that allows for the proliferation of target progenitor cells and seeded in any receptacle capable of sustaining cells, though as set out above, preferably in culture flasks or roller bottles. Cells typically proliferate within 3-4 days in a 37° C. incubator, and proliferation can be reinitiated at any time after that by dissociation or purification of the cells and re-suspension in fresh medium containing specific growth factors. The medium for cells suspension is also considered one type of cell carriers.

By “adipose” is meant any fat tissue. The adipose tissue may be brown or white adipose tissue, derived from subcutaneous, omental/visceral, mammary, gonadal, or other adipose tissue site. A convenient source of adipose tissue is from liposuction surgery, however, the source of adipose tissue or the method of isolation of adipose tissue is not critical to the invention. When stromal cells are desired for autologous transplantation into a subject, the adipose tissue will be isolated from that subject and administered to the specific breast defect site for tissue regeneration.

Any medium capable of supporting stromal cells in tissue culture may be used, for example, Dulbecco's Modified Eagle's Medium that supports the growth of fibroblasts. Growth factors are generally added to the medium for supporting stromal cells in tissue culture. Typically, 0 to 20% Fetal Bovine Serum (FBS) is added to the above medium in order to support the growth of stromal cells. The cells could be incubated at a temperature around 37° C. with the carbon dioxide content maintained between 1% to 10% and the oxygen content between 1% and 20%.

Non-limiting examples of media useful in the methods of the invention can contain fetal serum of bovine or other species at a concentration of at least 1% to about 30%, preferably at least about 5% to 15%, mostly preferably about 10%. Embryonic extract of chicken or other species can be present at a concentration of about 1% to 30%, preferably at least about 5% to 15%, most preferably about 10%.

The growth factors of the invention may include, but not limited to, transforming growth factor-β (TGF-β1, TGF-β2, TGF-β3 and the like), insulin-like growth factor, platelet derived growth factor, epidermal growth factor, acidic fibroblast growth factor, basic fibroblast growth factor, hepatocytic growth factor, and the like. The concentration of growth factors is about 1 to about 100 ng/ml. In one embodiment, the matrix for incorporating the stromal cells is a component of the collagenous extracellular matrix such as collagen I (particularly in the form of a gel). Other nutrient, such as vitamin A, vitamin A analogue (such as retinoic acid), vitamin B series, vitamin C, and vitamin C analogue or other vitamins may be added to the medium. The concentration of retinoic acid or other nutrient is about 0.1 to about 10 μg/ml.

The present invention also provides a method for formulating adipose derived stromal cells, either after in vitro culture or in absence of in vitro culture, with a biocompatible pharmaceutical carrier for injecting into the breast of a subject. In one embodiment, the biocompatible carrier may be in the form of slurry, gel, a malleable gel, colloid, solution, or suspension. A process for manufacturing an implantable cells-seeded gel material may comprise the steps of: providing a biocompatible carrier and stem cells source; combining the cells and the carrier in a uniformly suspended form; and applying a pressurizing force to the combined fluid for either injecting into the breast of the subject or for collapsing into a malleable gel before administering into the breast.

The adipose tissue derived stromal cells useful in the methods of invention may be isolated by a variety of methods known to those skilled in the art. For example, such methods are described in U.S. Pat. No. 6,153,432 incorporated herein in its entirety. In a preferred method, adipose tissue is isolated from a mammalian subject, preferably a human subject. A preferred source of adipose tissue is omental adipose. In humans, the adipose is typically isolated by liposuction. If the cells of the invention are to be transplanted into a human subject, it is preferable that the adipose tissue be isolated from that same subject so as to provide for an autologous transplant. Alternatively, the administered tissue may be allogenic.

In one embodiment of the invention, an adipose tissue derived stromal cell induced to express at least one phenotypic characteristic of a neuronal, astroglial, hepatic, hematopoietic, or breast tissue progenitor cell is provided. Phenotypic markers of the desired cells are well known to those of ordinary skill in the art, and copiously published in the literature. Additional phenotypic markers continue to be disclosed or can be identified without undue experimentation. Any of these markers can be used to confirm that the adipose cell has been induced to a differentiated state. Lineage specific phenotypic characteristics can include cell surface proteins, cytoskeletal proteins, cell morphology, and secretory products. Some aspects of the invention provide adipose tissue-derived stromal cells that exhibit the improved properties of increased extracellular matrix proteins and/or a lower amount of lipid than a mature isolated adipocyte.

Malson et al. in U.S. Pat. No. 4,772,419, entire contents of which are incorporated herein by reference, describes a crosslinked hyaluronic acid (or salt thereof) gel material that may be formed into a shaped article by pressure-drying or freeze-drying. The crosslinked hyaluronic material may be stored dry, and implanted or placed upon a body in dry form, or alternatively after being rehydrated in a saline solution. The crosslinking present in the material causes the material to be rehydrated as a sponge or foam, wherein the structure or shape is maintained, rather than forming a flowable hydrogel or putty. Some aspects of the invention provide a crosslinked gel material as a shaped article loaded with adipose-derived stem cells or progenitor breast tissue cells.

In another embodiment, the biocompatible cell carrier (for example, for cells to home in) or matrix may be a shaped construct, structure, or 3-dimensional scaffold. Examples of biocompatible carrier material includes alginate, agarose, fibrin, collagen, chitosan, gelatin, elastin, and combinations thereof. In one embodiment, the biocompatible cell carrier is biodegradable or bioresorbable. Examples of biodegradable matrix material may include, but not limited to, polymers or copolymers of lactide, glycolide, caprolactone, polydioxanone, and trimethylene carbonate. Examples of biodegradable matrix material may also include polyorthoesters and polyethylene oxide.

Further examples of biodegradable polymers for construction of the matrix may include aliphatic polyesters, alginate, cellulose, chitin, chitosan, collagen, copolymers of glycolide, copolymers of lactide, elastin, fibrin, glycolide/l-lactide copolymers (PGA/PLLA), glycolide/trimethylene carbonate copolymers (PGA/TMC), glycosaminoglycans, hydrogel, lactide/tetramethylglycolide copolymers, lactide/trimethylene carbonate copolymers, lactide/ε-capro- lactone copolymers, lactide/σ-valerolactone copolymers, l-lactide/dl-lactide copolymers, methyl methacrylate-N-vinyl pyrrolidone copolymers, modified proteins, nylon-2 PHBA/γ-hydroxyvalerate copolymers (PHBA/HVA), PLA/polyethylene oxide copolymers, PLA-polyethylene oxide (PELA), poly (amino acids), poly (trimethylene carbonates), poly hydroxyalkanoate polymers (PHA), poly(alklyene oxalates), poly(butylene diglycolate), poly(hydroxy butyrate) (PHB), poly(n-vinyl pyrrolidone), poly(ortho esters), polyalkyl-2-cyanoacrylates, polyanhydrides, polycyanoacrylates, polydepsipeptides, polydihydropyrans, poly-dl-lactide (PDLLA), polyesteramides, polyesters of oxalic acid, polyglycolide (PGA), polyiminocarbonates, polylactides (PLA), poly-l-lactide (PLLA), polyorthoesters, poly-p-dioxanone (PDO), polypeptides, polyphosphazenes, polysaccharides, polyurethanes (PU), polyvinyl alcohol (PVA), poly-β-hydroxypropionate (PHPA), poly-β-hydroxybutyrate (PBA), poly-σ-valerolact- one poly-β-alkanoic acids, poly-β-malic acid (PMLA), poly-ε-caprolactone (PCL), pseudo-Poly(Amino Acids), starch trimethylene carbonate (TMC), tyrosine based polymers. In another embodiment, the cell carrier or matrix functions as a reservoir for cell differentiation and controlled release to adjacent tissue sites.

Current protocols for differentiating isolated human preadipocytes into adipocytes can be performed by a variety of methods, for example, the preadipocyte cell component in human adipose tissue (the so-called “stromal vascular fraction” or SVF) can be isolated using collagenase treatment. The isolated human preadipocytes can then be driven to differentiate into adipocytes by a variety of chemical treatments. For example, Hauner's laboratory (Journal Clin Invest., (1989) 34:1663-1670) has shown that human preadipocytes can be induced to differentiate in serum-free medium containing 0.2 nM triiodothyronine, 0.5 μM insulin and 0.1 μM glucocorticoid. Similarly, it is disclosed in U.S. Pat. No. 4,153,432, entire contents of which are incorporated herein by reference, for the differentiation of human preadipocytes that incubating isolated human preadipocytes, plated at least about 25,000 cells/cm², in a medium containing, glucose, a cyclic AMP inducer such as isobutylmethylxanthine or forskolin, a glucocorticoid or glucocorticoid analogue, insulin or an insulin analogue and a PPARγ agonist or a RXR agonist.

### EXAMPLE NO. 1

### Methods of Transplantation

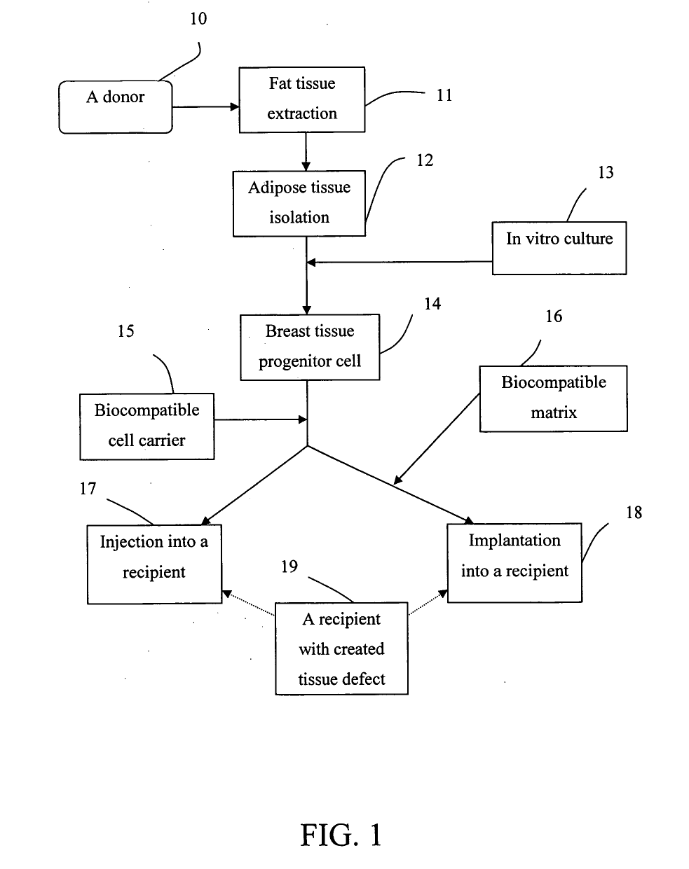

FIG. 1 shows a method of treating a breast defect in a patient, the method comprising: a) differentiating an isolated human adipose tissue derived stromal cell into a breast tissue progenitor cell; and b) administering the breast tissue progenitor cell to a breast defect area in the patient. In one embodiment, the fat tissue from the donor is further differentiated into adipocytes in an in vitro procedure, followed by isolation to obtain a concentrated substance of breat tissue progenitor cells prior to the step of administering. In one embodiment, the breast tissue defect is created as an adjunct step for promoting stem cells differentiation and tissue regeneration at about the defect site.

As shown in FIG. 1, the fat tissue extraction step 11 may be carried out, for example by liposuction from a donor 10. The adipose tissue isolation step 12 may include breakup of the fat mass and removal of the unwanted non-cellular material. In vitro culture step 13 may be optional; however, nutrients, growth factors and other substance may be added to enhance cell differentiation into breast tissue progenitor cells. In one embodiment, the breast tissue progenitor cells 14 can be formulated with biocompatible cell carrier 15 for injection into a recipient 17. In another embodiment, the breast tissue progenitor cells 14 can be further deposited onto a biocompatible matrix 16 for implantation into a recipient 18. It is one object of the present invention to provide a recipient 19 with created tissue defect enabling the stem cells tissue regeneration via the injection route 17 or the implantation route 18.

In another embodiment of the invention, support cells are used to promote the differentiation of the adipose-derived stromal cells prior to or following implantation into the defect breast site of a recipient. The support cells can be human or non-human animal derived cells. Adipose-derived cells are isolated and cultured within a population of cells; most preferably, the population is a defined population. The population of cells is heterogeneous and includes support cells for supplying factors to the progenitor cells of the invention. Support cells include other cell types that will promote the differentiation, growth and maintenance of the desired cells. By way of illustration, adipose-derived stromal cells are first isolated by any of the means described above, and grown in culture in the presence of other support cells. In another embodiment, the support cells are derived from primary cultures of these cell types taken from cultured human organ tissue. In yet another embodiment, the support cells are derived from immortalized cell lines. In some embodiments, the support cells are obtained autologously.

### EXAMPLE NO. 2

### Cell Carriers and Matrix

The formula consisting of breast tissue progenitor cells and cell carriers can be injected to the defect site of the breast using a syringe or other fluid delivery apparatus. In one embodiment, the formula is intended to enhance revascularization in situ. In another embodiment, the formula is intended to promote growth or multiplication of fat cells in the breast. For illustration purposes, the biocompatible matrix for cells to home in or adhere for intended differentiation purposes may comprise a foam or sponge that is compressible for inserting into the breast with a small opening. The biocompatible foam or sponge construct is characterized with plural pores, wherein at least a portion of the pores is interconnected and open to the outside of the construct. The foam or sponge can be cut, sized, and shaped as an implant. In one embodiment, the formula consisting of breast tissue progenitor cells and cell carriers may be loaded on the biocompatible matrix/foam before matrix/foam delivery into a recipient. Alternatively, the formula consisting of breast tissue progenitor cells and cell carriers may be injected to about the matrix/form site after the matrix/foam is implanted in place.

The gel or foam of the present invention may comprise methylcellulose, a temperature-sensitive polymer. Methylcellulose (MC) is a water-soluble polymer derived from cellulose, the most abundant polymer in nature. As a viscosity-enhancing polymer, it thickens solutions without precipitation over a wide pH range. A novel method using a temperature-sensitive polymer (Methylcellulose) to thermally gel aqueous alginate blended with distinct salts (CaCl₂, Na₂HPO₄, or NaCl), as a pH-sensitive hydrogel was developed for protein drug delivery (Biomacromolecules 2004;5:1917-1925). In the preparation of cells loaded hydrogels herein, it is suggested that stem cells is well-mixed to the dissolved aqueous methylcellulose or methylcellulose/alginate blended with salts at 4° C. and then gel by elevating the temperature to 37° C. In one embodiment, the blend (stem cells or adipose-derived breast tissue progenitor cells plus aqueous methylcellulose) is injected into the breast of a recipient and become a gel in situ because of the body temperature at 37° C., a characteristic temperature for methylcellulose.

All methylcellulose compositions exhibit the classical physical behavior of cellulose ethers, changing from a solution at lower temperature to a gel at elevated temperatures. When exposing methylcellulose to an increasing temperature, the methylcellulose shows an initial period of relatively constant viscosity. Then the solution undergoes an abrupt increase in viscosity at a characteristic temperature corresponding to initiation of the first gelation phenomenon. The temperature at which gelation is initiated can be altered by varying a number of factors, including concentration of methylcellulose polymer, formulation of the aqueous solvent, additives, and heating rate. Methylcellulose was reported biocompatible with little toxicity due to degraded byproducts (Biomaterials 2001;22:1113-1123). It was reported that injectable methylcellulose appears to be a suitable scaffold for bridging traumatically injured tissue when a cavity forms within the first few days following a traumatic insult to the cortex.

Poly(N-isopropyl acrylamide) demonstrated a fully expanded chain conformation below 32° C. and a collapsed compact conformation at high temperatures (J Biomed Mater Res 1993;27:1243-1251). In one aspect of the invention, adipose-derived breast tissue progenitor cells or stem cells are mixed with poly(N-isopropyl acrylamide) to form an injectable gel material. After loading the gel material into the breast of a recipient at adjacent the porous scaffold, the gel material collapses and squeezes into the pores of the scaffold, where the stem cells start differentiation and proliferation to repair or treat breast tissue defect.

The currently available breast augmentation devices include silicone breast implants filled with silicone fluid/gel or saline. Their disadvantages include: prone to rupture, prone to leak, losing the shape, or interfering with mammograms. Herein it is one object of the present invention to provide a breast implant that maintains its shape, will repair itself continuously, biocompatible, requires a minimally invasive intervention for implantation, and presents minimal or no complications because of its biocompatibility. In one embodiment, the breast implant of the present invention comprises a main shaping framework with sustained resorbable or biodegradable stent lattice, supplemented by a biodegradable or bioresorbable polymer netting. The material may include hydrogel with scaffold, matrix or foam configuration.

The delivery system of the presentation is catheter based. In one embodiment, the ability to repair itself continuously comprises delivering cell seeding slurry or concentrated cultured stem cells that is programmed to develop fat cells to augment the breast defect, wherein the cells may be derived from bone marrow stem cells or omentum fat cells.

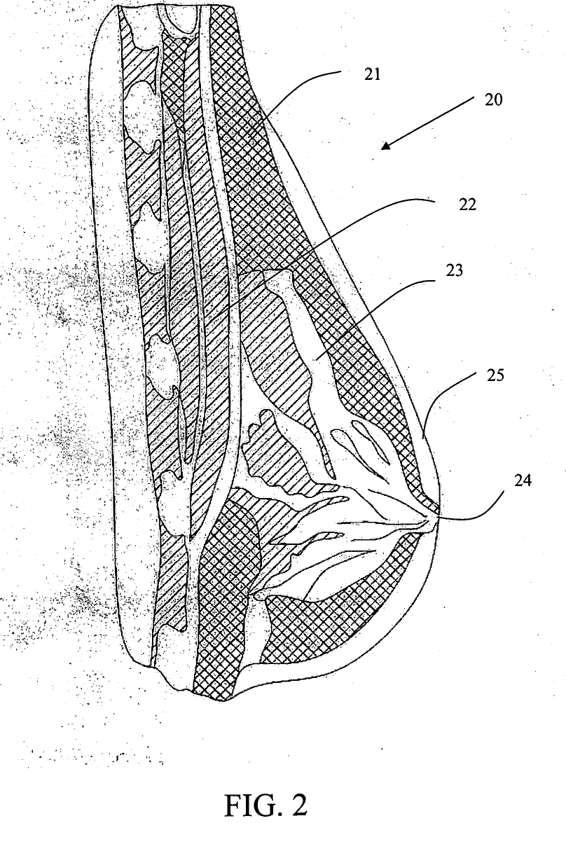

Some aspects of the present invention relate to various breast implant embodiments with stem cells loaded configuration or post-implantation stem cells receivable configuration and delivery systems thereof. FIG. 2 shows an anatomic illustration of a woman breast 20 comprising fatty tissue 21, muscle 22, ducts 23, and a nipple 24, among others. One embodiment of the present invention is to deliver a breast implant through the nipple and extendably follow the duct 23 or the space under the subcutaneous layer 25 of the breast. Another embodiment of the delivery route is similar to that of the silicone-gel breast implant placement.

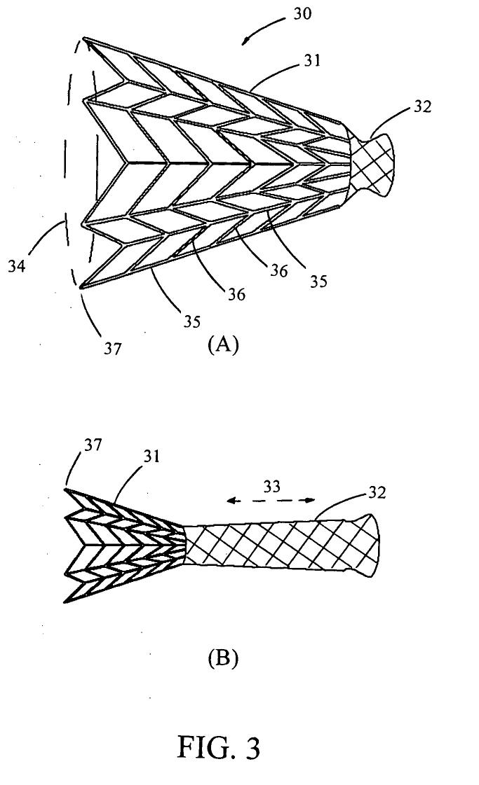

Various design configurations of the breast implant or scaffolds for stem cells seeding and eventual differentiation/regeneration/proliferation in situ are illustrated in FIGS. 3-7. FIG. 3 shows a breast implant embodiment of the fishbone design 30 at (A) an expanded profile, and (B) a collapsed profile. In general, the fishbone implant 30 comprises an expandable construct 31 with a plurality of close cells 38 formed between the longitudinal elements 35 and the connecting transverse elements 36, wherein the construct 31 is enclosed and loaded within the lumen of a sheath 32 during the initial delivery phase, the sheath being unobstructively movable substantially along the same direction 33 of the construct 31. In one embodiment, the sheath 32 is a solid cylinder, a meshed cylinder, or a flexible tubular apparatus that is detachable from the construct at the end of the delivery phase. The distal ends 37 of the construct 31 form a circular shape 34 after the implant is delivered to the breast site of a recipient. As discussed before, stem cells or adipose-derived breast tissue progenitor cells may be loaded on the breast implant before delivery or injected to adjacent the implant after the implant is delivered in place.

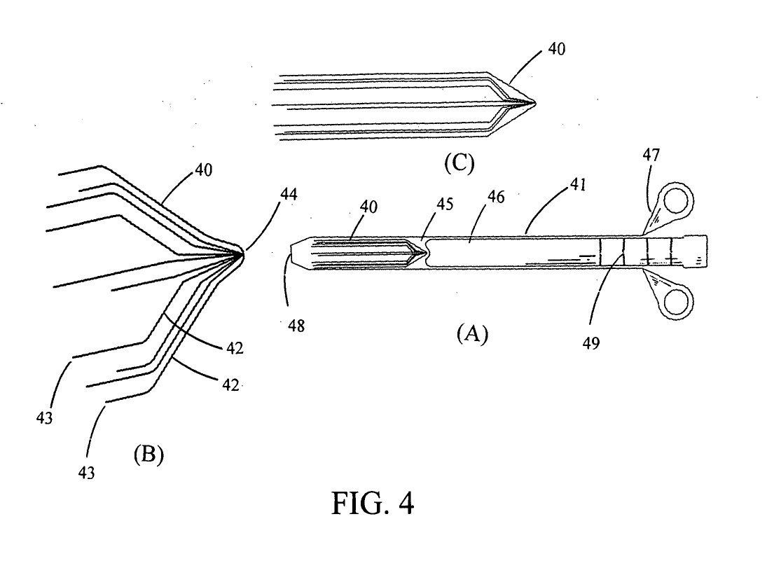

FIG. 4 shows a first breast implant embodiment of the umbrella design 40 with (A) a delivery instrument 41, (B) at an expanded device profile, and (C) at a collapsed device profile. In general, the umbrella implant 40 comprises a plurality of radially expandable extending elements 42, each having a distal end 43 and a proximal end 44. In one embodiment, the proximal ends 44 of all extending elements are secured together at one point. The delivery instrument 41 may comprise a lumen 45 with a pushing plunger 46, wherein the plunger is activated by a pushing mechanism 47 located at the handle of the delivery instrument. In operations, the needle tip 48 of the delivery instrument 41 contacts or partially penetrates the nipple of a recipient, the plunger is activated until the umbrella implant is fully deployed, which is indicated by a pre-marked marker 49 on the delivery instrument 41.

Some aspects of the invention provide a delivery instrument for delivering an umbrella-configured breast matrix to a breast of a patient comprising: a hollow tubular sheath having a distal needle tip, a lumen having an opening at the distal tip, and a handle portion; a plunger inside the lumen, wherein the plunger is activated by a pushing mechanism located at the handle portion; and wherein the lumen is sized and configured for appropriately receiving an umbrella-configured breast matrix at a collapsed profile.

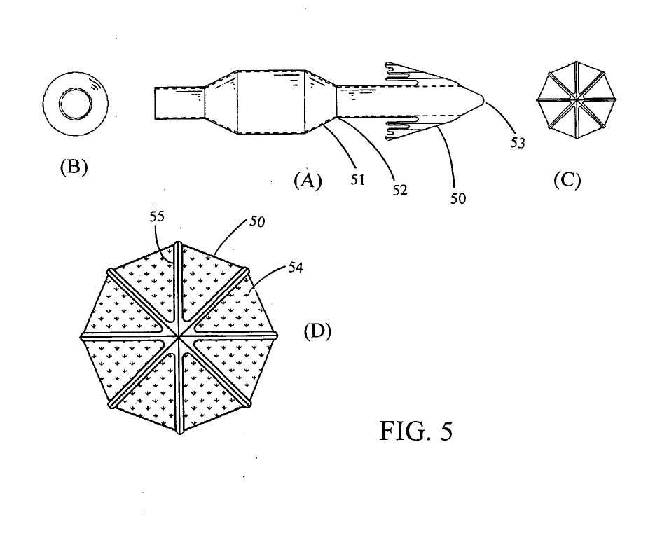

Alternatively, FIG. 5 shows a second breast implant embodiment of the umbrella design 50 with (A) a delivery instrument 51, (B) a proximal cross-sectional view of the delivery instrument, (C) a distal cross-sectional view of the delivery instrument, and (D) at an expanded device profile. Instead of loading the breast implant in the lumen of a delivery instrument as shown in FIG. 4, the breast implant 50 is loaded outside of the delivery instrument 51 in FIG. 5. In one embodiment, the second umbrella implant 50 comprises extending elements 55 and some connecting members 54 between the elements 55 to form an umbrella shape. The connecting member 54 may comprise netting, strings, threads, porous membranes, porous biodegradable films, biocompatible polymers, etc. as disclosed above. In operations, the instrument tip 53 of the delivery instrument 51 contacts and penetrates into the nipple of a recipient, the instrument 51 is pushed forward until the umbrella implant 50 at its collapsed profile is fully deployed inside the breast, which is indicated by a pre-marked marker 52 on the delivery instrument 51.

Some aspects of the invention provide a delivery instrument for delivering an umbrella-configured breast matrix to a breast of a patient comprising a tubular applicator having a distal tip, a distal portion and a handle portion, wherein the distal portion is sized and configured for appropriately receiving an umbrella-configured breast matrix at a collapsed profile over the distal portion.

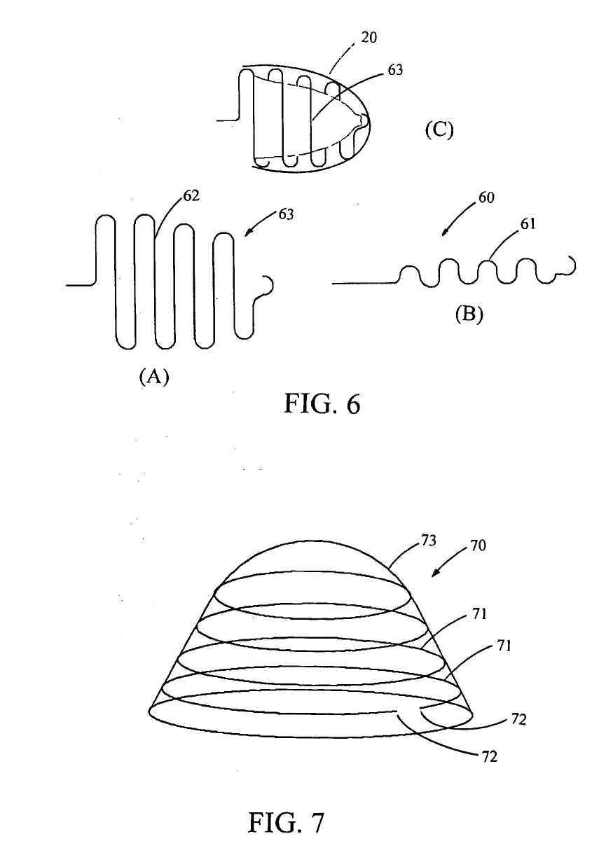

FIG. 6 shows a breast implant of the wrap-around design 60, (A) at an expanded profile, (B) at a collapsed profile, and (C) with a simulated profile. The wrap-around implant 60 may be made of shape memory polymer, shape memory biodegradable polymer or shape memory alloy, such as Nitinol. In one embodiment, a pre-shaped implant 60 is sized and shaped as a straight wire with a few small curvatures 61 at a first configuration (as shown in FIG. 6B). The implant is delivered into the breast, say from the nipple. After the implant is in place, the implant 60 is changed to a second 3-D configuration (as shown in FIG. 6A) with a few large curvatures 62 by the shape memory characteristics, mostly by raising the implant temperature to pass a shape transition temperature of the building material. In one embodiment, the shape transition temperature is configured to be a few degrees, preferably 1 to 5° C., above the body temperature. In operations, at least a major portion of the implant in the second configuration 63 is placed at a space under the subcutaneous layer of the breast (as shown in FIG. 6C) serving as a scaffold for stem cell deposition/differentiation leading to cell proliferation and tissue regeneration.

FIG. 7 shows a breast implant of the yo-yo design 70. In one embodiment, the implant comprises a plurality of circular rings 71 with varying diameters. In another embodiment, at least a portion of the circular rings 71 is an open ring with two ends 72 so that the ring can be inserted into the breast by first entering one end of the open ring into the nipple. In an alternate embodiment, at least two rings are releasably secured to each other by a circular semi-ring 73 to form a bowl-like configuration. In operations, each component (the circular rings or the circular semi-ring) of the yo-yo implant occupies certain locations of the breast for intended tissue regeneration by the loaded adipose-derived stem cells. In an alternative embodiment, the aforementioned breast implant is delivered to the breast by surgical operations or other penetration methods so that the implant serves as the supporting matrix for stem cells to repair or augment a breast tissue defect in a patient. The “breast implant” herein is intended to mean a scaffold, matrix, or stent to partially support the breast and partially support the loaded stem cells composition for tissue repair/augmentation, whereas the breast implant does not herein include or indicate any breast silicone prosthesis.

Noninvasive Growth Stimulation Devices

Unlike implantable Direct Current (DC) stimulation, external—or noninvasive— growth stimulation devices (particularly for bone growth stimulation) do not require surgical implantation. Rather, they are worn externally, either using:
Small, wafer-thin skin pads/electrodes that are placed directly over the target site and deliver Capacitive Coupling (CC) stimulation, or;
One or two treatment coil(s) delivering electromagnetic fields via Pulsed ElectroMagnetic Fields (PEMF's), or Combined Magnetic Fields (CMF), placed into a brace or directly onto the skin of the target site. One or two coils that generate an electromagnetic field at the target site are generally worn three to eight hours per day for three to six months after a spinal fusion or for bone growth stimulation.

The main drawback for this type of device is that the degree of patient compliance with the recommended treatment can hinder the clinical efficacy. If the patient does not wear the device, they will not receive the benefits from treatment. For noninvasive breast growth stimulation, some aspects of the invention provide a wearable electromagnetic generator apparatus to provide electromagnetic energy at about the surface of the target breast. In one embodiment, the generator apparatus or the treatment applicator (for example, the coil that generates an electromagnetic field or electrode/pads) of the electromagnetic energy generator apparatus is mounted on a bra (i.e., brassiere) or becomes an integral part of the bra construct or a breast support for delivering electromagnetic energy for intended breast tissue growth stimulation. In one embodiment, the treatment applicator is in intimate contact with the skin of the target site.

An exemplary bra structure with a massaging function that can be used in a method of the invention is described in U.S. Pat. No. 6,921,316 B1, which describes an apparatus that includes a body made of silicone rubber and having an outline for mating with that of a female bosom. The body includes a reinforcing rib formed along a perimeter thereof. A rear member is mounted to an inner side of the body, wherein the rear member includes a plurality of protrusions formed on an outer face thereof and are in contact with the female bosom for massaging the female bosom.

Electromagnetic Growth Stimulation

As used herein, the term “electromagnetic energy” is intended to mean a form of energy having both electric and magnetic components and properties of wavelength and frequency. Forms of energy included in the term are, for example, X-ray radiation, which has a wavelength in the range of about 0.05 to 100 angstroms; ultraviolet radiation, which has a wavelength in the range of about 200 to 390 nm; visible radiation, which has a wavelength in the range of about 391 to 770 nm; infrared radiation, which has a wavelength in the range of about 0.771 to 25 microns; microwave radiation, which has a wavelength in the range of about 1 millimeter to 1 meter; and radiofrequency radiation, which has a wavelength greater than about 1 meter.

As used herein, the term “electromagnetic stimulation” means any form of electromagnetic energy including, but not limited to, electromagnetic radiation or pulsed electromagnetic field stimulation (PEMF).

As used herein, the term “stimulating growth” is intended to mean initiating or increasing the rate at which cells proliferate. The term can include, for example, accelerating the cell cycle, initiating entry into the cell cycle, or leaving G₀ or the resting state. As used herein, the term “tissue” is intended to mean a group of cells united to perform a particular function.

U.S. Pat. No. 6,190,893, issued on Feb. 20, 2001, entire contents of which are incorporated herein by reference, discloses compositions, methods and systems provided for the stimulation of biological activities within bone marrow stromal cells by applying electromagnetic stimulation to an electroactive material, wherein the electromagnetic stimulation is coupled to the electromagnetic material.

The electroactive material may be in a spherical form (for example, microparticles or nanoparticles) for injecting into a target breast via a syringe needle or a microprojectile bombardment process as described below. The implanted electroactive material receives energy remotely via electromagnetic or ultrasound transmission to enhance growth stimulation. One aspect comprises the metallic electroactive particles selected from the group consisting of gold, silver, platinum, tungsten, stainless steel, titanium, and the like. In one embodiment, the metallic microparticles are about one micro in size.

Another aspect comprises the conductive polymeric particles for injecting into a target breast via a syringe needle or a microprojectile bombardment process selected from the group consisting of polypyrrole, poly(p-phenylene), poly(p-phenylene-vinylene), poly(thiophene), poly(aniline), poly(porphyrin), and poly(heme). The electroactive polymers suitable for use in the present invention include a new class of organic polymers with a remarkable ability to conduct electrical current. These electrically conducting polymers typically possess a conjugated backbone with a high degree of p-orbital overlap. Through a process known as “doping”, the neutral polymer can be oxidized or reduced to become either positively charged (oxidative, p-type) or negatively charged (reductive, n-type). The generation and propagation of charge occurs via polarions or bipolarions along the oxidized polymer backbone. The conductive form of the polymer contains counterions that serve to maintain charge neutrality but do not affect the oxidation level of the polymer. In one embodiment, the polymeric microparticles are about one micro in size.

Some aspects of the present invention provide methods for the stimulation of biological activities within cells, which involves associating the desired cells to indwelling electroactive microparticles, and applying electromagnetic stimulation directly to the desired cells. In preferred embodiments, the stimulation of biological activities within cells results from inducing one or more activities including, but not limited to, gene expression, cell growth, cell differentiation, signal transduction, membrane permeability, cell division, and cell signaling.

In one preferred embodiment, the present invention provides a method for stimulating one or more biological activities of cells comprising contacting cells with an electroactive substrate, wherein the electroactive substrate comprises breast implants of the present invention; for example, a breast implant embodiment of the fishbone design 30 in FIG. 3, a breast implant embodiment of the umbrella design 40 in FIG. 4, a breast implant embodiment of the umbrella design 50 in FIG. 5, a breast implant of the wrap-around design 60 in FIG. 6, and a breast implant of the yo-yo design 70 in FIG. 7. The material for the breast implants 30, 40, 50, 60, and 70 may be a conductive material such as stainless steel, god, silver, nitinol or the conductive polymers.

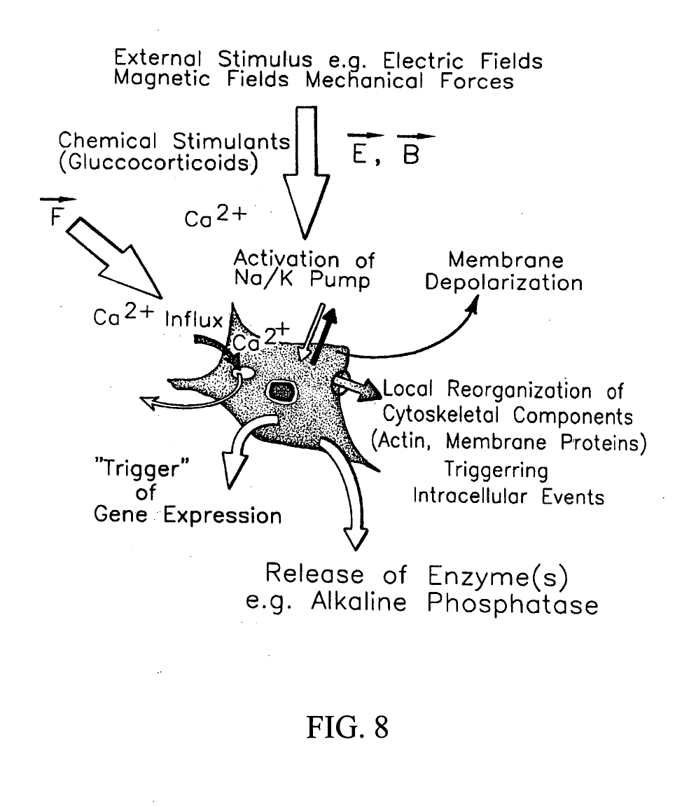

As previously illustrated in U.S. Pat. No. 6,190,893, FIG. 8 depicts the effect of an external stimulus on one or more biological activities within cells by a variety of mechanisms, such as, but not limited to, conformational changes in readsorbed proteins on the electroactive substrate upon electromagnetic stimulation, by electrophoretic redistribution of cytoskeletal components, by activation of voltage gated Ca²⁺ and Na/K ion channels, and by depolarization of membrane resting potentials. The biological activities within the cell are gene expression, cell growth, cell differentiation, cell signal transduction, and cell signaling. The cells to be stimulated may also comprise stem cells (particularly the stem cells derived from adipose tissue) that are characterized in that they are not themselves terminally differentiated, they can divide without limit, and when they divide, each daughter cell has the choice of either remaining a stem cell, or embarking on a course leading irreversibly to terminal differentiation to become a breast tissue progenitor cell or breast adipose-like cells.

The tissue growth stimulation may also be effected by other means for contacting or applying pressure/pulsed pressure to the tissue from the exterior surface of the breast. This may include pneumatic force, mechanical force, low voltage electric, magnetic force, acupressure, or acupuncture at the appropriate points of areas of the breast.

In the case of noninvasive stimulators, external electromagnetic coils are placed on the surface of the target breast and are held in place by a strap or cuff or the breast support. Locating the coils correctly is important. The coils produce a pulsating electromagnetic field. It is up to the patient to maintain the prescribed treatment schedule. Effective treatment requires stimulation anywhere from three to ten hours each day in periods of no less than one hour. Ultrasound stimulation is the most recent treatment for stimulating tissue or bone growth. A device that generates low intensity pulses of sound is applied to the skin. The advantage of this technique is that it is noninvasive and the period of application of the sound pulses can be as short as 20-30 minutes each day.

U.S. Pat. Appl. Publication No. 2002/0009797 to Wolf et al., entire contents of which are incorporated herein by reference, discloses a system for growing mammalian cells within a culture medium facilitated by an electromagnetic field, and preferably, a time varying electromagnetic field. The electrical current for generating the electromagnetic field is from about 1 mA to about 1,000 mA. It further discloses that the time varying electromagnetic field induces a cellular response at gene level, wherein the cellular response is cellular control of growth and differentiation at gene level, and wherein the cellular control of growth and differentiation is to suppress or enhance growth regulatory functions at gene level.

U.S. Pat. Appl. Publication No. 2005/0059153 to George et al., entire contents of which are incorporated herein by reference, discloses a method for activating a cell cycle regulator by delivering to a cell an effective amount of electromagnetic energy. It also discloses a method with electromagnetic energy for activating a signal transduction protein; a method for activating a transcription factor; a method for activating a DNA synthesis protein; a method for activating a Receptor; and a method for replacing damaged neuronal tissue as well as a method for stimulating growth of administered cells.

Some aspects describe electromagnetic energy delivered to a cell using any apparatus capable of generating and applying known dosages of electromagnetic energy of defined specifications to the cell. Generally, an apparatus useful in the invention for delivering electromagnetic energy to a cell will include an electromagnetic energy generator, a treatment applicator that delivers energy from the generator to a cell and a device (for example, a controller) for controlling the amount or characteristics of the electromagnetic energy delivered by the applicator. An exemplary electromagnetic energy treatment apparatus that can be used in a method of the invention is described in U.S. Pat. No. 6,344,069 B1, which describes an apparatus that includes a pulsed electromagnetic energy generator, a power controller, including a power level controller responsive to signals from multiple sensing and control circuits, and a treatment pad applicator.

One aspect describes the parameters under which electromagnetic energy is delivered to a cell being adjusted to suit a particular application of the methods. Exemplary parameters that can be adjusted include, without limitation, wavelength, power level, duration of delivery, delivery of constant output or pulsed output and, if pulsed output is used, pulse rate and pulse width. Typically, the electromagnetic energy is delivered under parameters in which the cell being treated does not sustain substantial DNA damage. Another aspect describes one parameter that can be adjusted being the number of electromagnetic energy deliveries given to a cell during a specified time period. Electromagnetic energy can be delivered in a single administration or in multiple deliveries. Multiple deliveries can be administered over a time period of minutes, hours, days or weeks. The parameters for delivery of electromagnetic energy for a particular application of the methods can be determined based on a dose-response analysis. Those skilled in the art will know or be able to determine an appropriate response that indicates a favorable outcome for a particular application such as treatment of a disease or condition and will be able to systematically vary the parameters while evaluating the response as it correlates with a desired outcome.

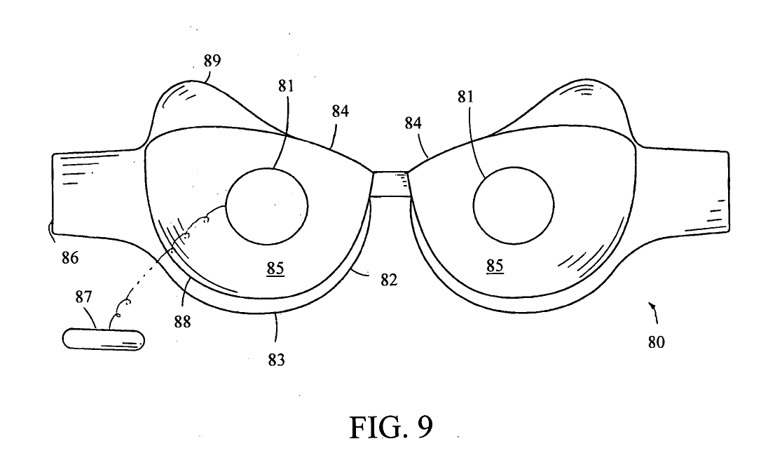

Some aspects of the invention provide that the electromagnetic energy or the capacitative coupling stimulation means is delivered from a treatment applicator mounted on a bra or a breast support, the treatment applicator delivering the electromagnetic energy or the capacitative coupling stimulation from the electromagnetic energy generator to the cells. FIG. 9 shows a bra apparatus having capability for electromagnetic stimulation functions. The bra 80 has a pair of cups 84, preferably molded of silicon, having an interior surface 85 facing the skin of a breast and an exterior surface facing the outer cloths. The cups 84 are substantially oval in shape and are slightly contoured to fit intimately over and cover the breasts of the user. The cups 84 include a periphery 88. A flange 83 is attached to each of the cups 84 by extending outwardly from a portion of the periphery 88 of the cups 84. The flanges 83 each have a first surface 82, an upper tab 89, and a side tab 86. The flange 83 for each cup 84 extends outwardly from a portion of the periphery 88 of that cup 84. A treatment applicator 81 are attached to the interior surface 85 of each of the cups 84 for providing electromagnetic energy to the breast when the bra 80 is worn by the user and the energy is activated. The treatment applicator is connected to a remote energy generator 87 for supplying the needed dosage of electromagnetic energy. In one embodiment, the power controller is located in a pocket on the cloths for the patient to start/stop the stimulation process or to adjust the stimulation dosage as needed/prescribed.

Some aspects of the invention further provide a method for stimulating growth of administered cells. The method includes the steps of (a) administering a population of cells to an individual, and (b) delivering to the population an effective amount of electromagnetic energy to stimulate growth of the population. In one embodiment, the population of cells can be administered to a site of tissue damage, such as those described above, and stimulated to replace the damaged tissue. A population of cells can also be administered in a method of treating other defects in the body such as the deficiency or over abundance of a particular gene product. Accordingly, a method of the invention can include administering cells that either naturally express an effective amount of a gene product for a desired therapeutic effect or that have been genetically manipulated to do so using, for example, the methods described herein above.

A cell or population of cells administered to an individual in a method of the invention can be any type of cell that is appropriate for replacing a tissue or performing a desired function including, for example, those set forth above. A population of cells that is administered in a method of the invention can be in a tissue or organ that is isolated as a tissue or organ from a donor individual or that is produced in a culture system.

For therapeutic applications, the above cell types are additionally chosen to remain viable in vivo without being substantially rejected by the host immune system. Therefore, the donor origin of the cell type should be evaluated when selecting cells for therapeutic administration. A cell can be autologous, wherein it is administered to the same individual from whom it was removed or can be heterologous being obtained from a donor individual who is different from the recipient individual. Those skilled in the art know what characteristics should be exhibited by cells to remain viable following administration. Moreover, methods well known in the art are available to augment the viability of cells following administration into a recipient individual.

Microprojectile Bombardment Process

In general, “Gene gun” is a device that delivers DNA to cells by a microprojectile bombardment process with extremely high-speed delivery. The Helios® Gene Gun has been a new way for in vivo transformation of cells or organisms (i.e. gene therapy, genetic immunization, or DNA vaccination). This gun uses Biolistic® particle bombardment where DNA- or RNA-coated gold particles are loaded into the gun and one pulls the trigger for delivery. A high-pressure helium pulse delivers the coated gold particles into virtually any target cell or tissue. The particles carry the DNA so that one does not have to remove cells from tissue in order to transform the cells.

One model of the Helios gene gun system, 220-240 V, is used for biolistic particle delivery of biomaterials into cells. This handheld device employs an adjustable helium pulse to sweep DNA-, RNA-, and other biomaterial-coated gold microcarriers from the inner wall of a small plastic cartridge directly into target cells. This system has a 2 square-centimeter target area, and uses a pressure range of 100-600 psi. The system includes the Helios gene gun, helium hose assembly, helium regulator, tubing prep station, syringe kit, tubing cutter, and Helios gene gun optimization kit. Dimensions are 20×25 cm (manufactured by Bio-Rad, Hercules, Calif.). The gene gun is a device for injecting cells with genetic information, originally designed for plant transformation. The payload is an elemental particle of a heavy metal coated with plasmid DNA. The actual name of the gene gun is the Biolistic Particle Delivery System, and this technique is often simply referred to as “biolistics”—a cross between biology and ballistics.

In some aspects, another model of the gene gun consists of two small 6″×7″×10″ stainless steel chambers connected to a 2 HP vacuum pump. When the technician flicks the switch on the outside of the second chamber, helium is released at up to 1000 psi. The blast ruptures a first disk about the size of a nickel. The explosion of the first disk releases a shock wave which travels 1 centimeter until it hits a second disk, which is free to move. Attached to the front of that second disk are microscopic tungsten or gold particles 1 micron in diameter coated with thousands of DNA molecules. This second disk travels another centimeter at the speed of a rifle bullet, for example about 1300 feet per second, and hits a screen, which detains the second disk, but launches the microscopic particles toward the target cells. The particles penetrate the cells and release the DNA, which is diffused into the nucleus and incorporated by the chromosomes of the plant. One very common way of introducing DNA into plant cells is through DNA coated particles (e.g. one micron gold particles) that are literally shot through the cell wall. The gene gun was originally a nail gun for concrete surfaces modified to fire tungsten particles. Later the design was greatly refined. Improvements include the use of helium propellant and a multi-disk-collision delivery mechanism. Other heavy metals such as gold and silver are also used, but not as frequently due to reasons of availability and cost. The gene gun is very useful in applications such as transfection in agriculture, gene therapy or gene vaccine.

Another model of microprojectile bombardment gun is the “Cloning Gun™” that is a cordless, rechargeable, hand-held electroporation instrument. A cloning gun generally achieves transfection efficiencies exceeding 50% of viable cells with a variety of standard mammalian cell lines.

An exemplary microprojectile bombardment gene gun apparatus that can be used in a method of the invention is described in U.S. Pat. No. 6,194,389 B1, which describes a currently available microprojectile bombardment apparatus, comprising a bombardment chamber shown with a stopping plate and a delivering chamber with a sealing plate positioned for injecting to the subject.

Some aspects of the invention relate to a method of administering a bioactive agent or conductive microparticle in an animal subject by in situ microprojectile bombardment, comprising: (a) selecting a target breast skin tissue of the subject, wherein the target skin tissue is selected from the group consisting of epidermis tissue, dermis tissue, and hypodermis tissue; (b) providing microprojectiles; and (c) accelerating the microprojectiles at the subject so that the microprojectiles contact the epidermis at a speed sufficient to penetrate the epidermis and lodge in the target tissue.

Although the present invention has been described with reference to specific details of certain embodiments thereof, it is not intended that such details should be regarded as limitations upon the scope of the invention. Many modifications and variations are possible in light of the above disclosure.

What is claimed is:

1. A method for stimulating growth of administered cells in a breast, comprising the steps of:
(a) administering a population of stem cells to the breast of a subject, wherein the stem cells are derived from adipose tissue; and
(b) delivering to the population an effective amount of electromagnetic energy from an electromagnetic energy generator to stimulate growth of the population.

2. The method according to claim 1, wherein the electromagnetic energy is delivered with an electromagnetic field or a pulsed electromagnetic field.

3. The method according to claim 1, wherein the electromagnetic energy is X-ray radiation, which has a wavelength in the range of about 0.05 to 100 angstroms.

4. The method according to claim 1, wherein the electromagnetic energy is ultraviolet radiation, which has a wavelength in the range of about 200 to 390 mn.

5. The method according to claim 1, wherein the electromagnetic energy is visible radiation, which has a wavelength in the range of about 391 to 770 nm.

6. The method according to claim 1, wherein the electromagnetic energy is infrared radiation, which has a wavelength in the range of about 0.771 to 25 microns.

7. The method according to claim 1, wherein the electromagnetic energy is microwave radiation, which has a wavelength in the range of about 1 millimeter to 1 meter.

8. The method according to claim 1, wherein the electromagnetic energy is radiofrequency radiation, which has a wavelength greater than about 1 meter.

9. The method according to claim 1, wherein the method further comprises delivering at least one conductive microparticle into the breast configured for relaying the electromagnetic energy to stimulate the growth of the population.

10. The method according to claim 9, wherein the conductive microparticle is selected from the group consisting of gold, silver, platinum, tungsten, stainless steel, and titanium.

11. The method according to claim 9, wherein the conductive microparticle is selected from the group consisting of polypyrrole, poly(p-phenylene), poly(p-phenylene-vinylene), poly(thiophene), poly(aniline), poly(porphyrin), and poly(heme).

12. The method according to claim 9, wherein the conductive microparticle is about one micron in size.

13. The method according to claim 9, wherein the step of delivering the at least one conductive microparticle is by a microprojectile bombardment process.

14. The method according to claim 13, wherein the microprojectile bombardment process comprises: (a) selecting a target breast skin tissue of the subject, wherein the target skin tissue is selected from the group consisting of epidermis tissue, dermis tissue, and hypodermis tissue; (b) providing microprojectiles of the conductive microparticles; and (c) accelerating the microprojectiles at the subject so that the microprojectiles contact the epidermis at a speed sufficient to penetrate the epidermis and lodge in the target tissue.

15. The method according to claim 9, wherein the step of delivering the at least one conductive microparticle is by a syringe needle.

16. The method according to claim 1, wherein the electromagnetic energy is delivered from a treatment applicator mounted on a bra, said treatment applicator delivering the electromagnetic energy from the electromagnetic energy generator to the population of cells.

17. The method according to claim 1, wherein the stem cells comprise breast tissue progenitor cells.

18. A method for stimulating growth of administered cells in a breast, comprising the steps of:
(a) administering a population of cells to the breast of an individual; and
(b) delivering to the population an effective amount of electric energy via capacitative coupling stimulation means for stimulating growth of the population.

19. The method according to claim 18, wherein the capacitative coupling stimulation means is delivered from a treatment applicator mounted on a bra.

20. A method for stimulating one or more biological activities of cells comprising contacting the cells with an electroactive substrate, wherein the electroactive substrate comprises a breast implant made of conductive material.
